(19) United States
(12) Patent Application Publication
(10) Pub. No.: US 2010/0240076 A1
(43) Pub. Date: Sep. 23, 2010

(54) IMMUNOASSAY INVOLVING MUTANT ANTIGENS TO REDUCE UNSPECIFIC BINDING

(75) Inventors: Uwe Jacob, Muenchen (DE); Constanze Breithaupt, Jena (DE); Robert Huber, Muenchen (DE)

Correspondence Address: Baker Donelson Bearman, Caldwell & Berkowitz, PC, 920 Massachusetts Ave, NW, Suite 900, Washington, DC, 20001, US

(73) Assignee: MAX-PLANCK-GESELLSCHAFT ZUR FOERDERUNG DER WISSENSCHAFTEN E.V.

(21) Appl. No.: 12/376,774

(22) PCT Filed: Jul. 12, 2007
(86) PCT No.: PCT/EP07/06217

§ 371 Date: May 13, 2010

(30) Foreign Application Priority Data
Aug. 8, 2006 (EP) EP06016554.5

(51) Int. Cl. G01N 33/53

(52) U.S. Cl. 435/7.92; 436/501; 435/7.1

(57) ABSTRACT

A method for a quantitative in vitro analysis to diagnose, to categorise, to predict and/or to monitor the progression of a condition comprising the following steps: a) Obtaining a sample suspected of containing anti-A-antibodies from a subject to be analysed, b) Providing native and mutant antigen A, c) Contacting the sample suspected of containing anti-A-antibodies with mutant antigen A and with native antigen A, d) Detecting the amount of anti-A-antibodies bound to native antigen A after step c), wherein the presence of anti-A-antibodies bound to native antigen A allows the diagnosis, the categorisation, the prediction and/or the monitoring of the progression of a condition.

The present invention concerns in general the field of antigen-antibody-interaction-based analysis-methods and kits therefore. In particular, the present invention concerns a method for a quantitative in vitro analysis to diagnose, to categorise, to predict and/or to monitor the progression of a condition in accordance with claim 1 and a kit for carrying out such a method in accordance with claim 26.

Antigens are large molecules, usually proteins, viruses, fungi, bacteria, and also substances such as toxins, chemicals, drugs, and other particles that are foreign to an organism. The immune system recognizes antigens and produces antibodies as a part of the humoral immune response.

An antibody is a protein used by the immune system to identify and neutralise antigens. During an immune response against specific antigens antibodies evolve that specifically binds to these antigens.

Antibodies can be anchored to the cell membrane of immune cells or they can exist freely in the blood and in tissue fluids, as well as in many secretions. Free antibodies have two primary functions:
combining with specific immunoglobulin receptors and exerting effector functions, and
binding to antigens and crosslinking them.

In binding to antigens, they can cause agglutination and precipitation of antibody-antigen products primed for phagocytosis by macrophages and other cells, block viral receptors, and stimulate other immune responses, such as the complement pathway.

Because antibodies are generated by the humoral immune system of the body almost immediately after detection of the presence of antigens, they usually appear at a very early stage of development of a condition.

This early appearance makes the detection of antibodies in theory an attractive tool to diagnose a condition early.

Because of the antigen specificity of antibodies, the detection of specific antibodies is used in medical diagnostics.

Serology depends on these methods. Autoimmune disorders sometimes can be traced to antibodies that bind the body's own proteins; a few can even be detected through blood tests. Antibodies directed against RBC surface antigens in immune mediated hemolytic anemia can be detected with the Coombs test. The Coombs test is also used for antibody screening in blood transfusion preparation and also for antibody screening in antenatal women.

However, problematic with all these approaches is that in general the kinds and the amounts of antibodies present in the immune systems of two individuals are hardly comparable.

One field where such an early diagnostics tool would be highly desirable is the diagnosis of cancer and autoimmune disorders.

Cancer results when cells lose their response to growth regulatory pathways and multiply abnormally. This uncontrolled outgrow is connected to evolution and abnormal expression patterns of gene products, which often results in immune recognition and antibody production of the body against certain tumor specific (tumor marker) structures. Clearly, measurement of the antibody appearance against tumor markers could lead to early diagnosis of cancer or determination of the progression and prognosis of cancer.

Autoimmune disorders are conditions caused by an immune response against the body's own tissues. This is caused by a hypersensitivity reaction similar to allergies, where the immune system reacts to a substance that it normally would ignore. In allergies, the immune system reacts to an external substance that would normally be harmless. With autoimmune disorders, the immune system reacts to normal “self” body components.

Normally, the immune system is capable of differentiating “self” from “non-self” tissue. Some immune system cells (lymphocytes) become sensitized against “self” tissue cells, but these faulty lymphocytes are usually removed or controlled (suppressed) by other lymphocytes. Autoimmune disorders occur when the normal control process is disrupted. They may also occur if normal body tissue is altered so that it is no longer recognised as “self.”

An autoimmune disorder may affect only one organ or tissue type or may affect multiple organs and tissues. Organs and tissues commonly affected by autoimmune disorders include blood components such as red blood cells, blood vessels, connective tissues, endocrine glands such as the thyroid or pancreas, muscles, joints, and skin.

One example of an autoimmune disorder is multiple sclerosis (MS).

MS is a central nervous system disorder marked by decreased nerve function with initial inflammation of the protective myelin nerve covering and eventual scarring. Symptoms and severity of symptoms vary widely and often progress into episodes of crisis alternating with episodes of remission.

It was discovered that myelin oligodendrocyte protein (MOG), that is expressed exclusively in the central nervous system (CNS), is the immunodominant target of demyelinating auto antibodies in the guinea pig model of experimental autoimmune encephalomyelitis (EAE), the animal model of MS (Lebar, R., et al., 1986, Clinical and Experimental Immunology, 66:423-34; Linnington, C., et al., 1984, Journal of Neuroimmunology, 6:387-96).

The pathogenic role of antibodies targeting MOG in EAE and the exposed location of MOG at the outermost lamella of CNS myelin indicate that MOG may also act as important auto antigen in MS, as evidenced by the detection of MOG-specific antibodies in the CNS tissue of MS patients (O'Connor, et al., 2001, Journal of Clinical Immunology, 21:81-92).

However, no clear evidence exists about the presence of MOG-specific antibodies in serum or in the cerebrospinal fluid (CSF) of MS patients. Several laboratories have attempted to detect these anti-MOG antibodies with quite differing results.

While some laboratories detect significantly elevated anti-MOG antibody levels (De March, A. K. et al., 2003, Journal of Neuroimmunology, 135:117-125; Gaertner, S. et al., 2004. Neurology, 63:2381-2383; Iglesias et al., 2001, Glia, 36:220-234; Berger, et al., 2003, New England Journal of Medicine, 349:139-145) others measure similar concentrations in patients with other inflammatory neurological diseases or even in healthy controls (Haase, et al., 2001, Journal of Neuroimmunology, 114:220-225; Lampasona et al., 2004, Neurology, 62:2092-2094; Lim, et al., 1986, Journal of Biological Chemistry, 261:5140-5146). These results were in general obtained by either using ELISA or RIA techniques.

This discrepancy was attributed to differences in the selection of patients and assay performance.

The amounts and kinds of antibodies present in the immune system of a subject to be analysed varies considerably based on a number of factors such as its race, sex, area of living, lifestyle, age, previous antigens encountered, inheritance, other present diseases or nutrition. These individual variations may render the detection of specific antibodies impossible when the level of these antibodies is low and/or the unspecific background is high.

Nevertheless, auto antibodies often appear a long time before the first symptoms of a condition become evident and an early diagnosis of autoimmune diseases is highly desirable to guarantee an optimal therapy.

Yet, today an early diagnosis of conditions such as, e.g., autoimmune disorders, in particular MS is extremely difficult, a prediction with respect to the progression of such a condition is next to impossible.

Using this early appearance as an analytical tool could help to drastically increase the success rate for the treatment of these conditions and in some instances could even help to prevent that symptoms ever appear.

In addition, early detection of autoantibodies could help to determine subtypes of a disease. MS patients are categorized into four groups depending on the type of the immune reaction that dominates. In Type II MS the progression of the disease is dependent on auto-antibodies against constituents of the Myelin sheath. Since these patients usually benefit from specific therapies like IVIG, Rituxan or Plasmapheresis, it would be highly desirable to diagnose these subgroup of patients early and convey them to their effective therapy.

In an attempt to use the appearance and specificity of antibodies as an analytical tool and to overcome the above mentioned and other disadvantages and problems of the present state of the art the present inventors have completed the following invention.

It was the object of the present invention to provide a fast, simple and easy-to-use method for a quantitative in vitro analysis to diagnose, to categorise, to predict and/or to monitor the progression of a condition based on antibody-antigen interactions, that overcomes or at least reduces the problems associated with the methods of the prior art, in particular that overcomes or at least reduces the problems associated with the individual properties of each subject to be analysed, in particular the amounts and kinds of antibodies present in its immune system, and its incomparability with other subjects.

This object is solved by a method in accordance with claim 1-25.

It was a further object of the present invention to provide the state of the art with a kit that contains all necessary parts to carry out the method of the present invention.

This object is solved by a kit in accordance with claim 26-33.

Those skilled in the art will understand that it is possible to freely combine any features of the present invention disclosed herein. This will result in further embodiments of the present invention, that are considered to be comprised by its scope.

It is furthermore referred to all references cited herein. Their relevant content is to be considered a part of the disclosure of the present invention.

The method of the present invention is a method for a quantitative in vitro analysis to diagnose, to categorise, to predict and/or to monitor the progression of a condition.

The diagnosis, preferably an early diagnosis, of a wide variety of different conditions is one field of application of the method of the present invention. Most disorders of an organism are reflected at a very early state in the humoral immune system of the corresponding subject. Detecting the presence of specific antibodies for antigens that cause a condition reliably is therefore a powerful tool to diagnose a condition, preferentially even before symptoms of the condition appear. As it is commonly known, it is of a significant value in medicine, to be able to diagnose a condition early. The method of the present invention can be applied after symptoms of the condition have appeared to provide further evidence to safely diagnose the condition, but equally well also before the appearance of any symptoms at all with apparently healthy individuals in the framework of, e.g., regular and/or irregular medical check-ups. The method of the present invention is also applicable after the death of a subject, e.g., to determine its cause of death or to determine any other disorders the dead subject might have suffered from.

The categorisation of a condition, in particular of disorders, is another important field of application for the method of the present invention. Oftentimes a single disorder with its symptoms can be the result of differing underlying biochemical or physiological causes. In order to be able to advise a correct therapy it is therefore crucial, to determine the cause of the disorder correctly. The method of the present invention allows it to discriminate between different types of a disorder even though the symptoms might be identical for all types of that disorder.

The method of the present invention can also be applied for the correct prediction of the progression of a condition, in particular of a disorder. Such a correct prediction allows to choose the appropriate therapy. It furthermore adds to the atmosphere of trust between medical practitioner and the patient and avoids, that the patient does not know what to expect in the future. Appropriate preparations can be made in time.

Finally, the monitoring of a condition is another application example of the subject matter of the present invention. This application allows it for example, that the effectiveness of a medication is checked after a relatively short time after application a medication, a long time before symptoms of healing can be expected to show. This allows to abort ineffective medication early, while avoiding a time loss and inadvertent and unnecessary side effects, and also allows to detect the effectiveness of a medication early, which will add to the comfort of a patient.

Subject matter of the present invention is a method for a quantitative in vitro analysis to diagnose, to categorise, to predict and/or to monitor the progression of a condition comprising the following steps:

a) Obtaining a sample suspected of containing anti-A-antibodies from a subject to be analysed,
b) Providing native and mutant antigen A,
c) Contacting the sample suspected of containing anti-A-antibodies with mutant antigen A and with native antigen A,
d) Detecting the amount of anti-A-antibodies bound to native antigen A after step c)
wherein the presence of anti-A-antibodies bound to native antigen A allows the diagnosis, the categorisation, the prediction and/or the monitoring of the progression of a condition.

Optionally, the sample suspected of containing anti-A-antibodies from a subject to be analysed can be first brought into contact with mutant antigen A. Further optionally, the complexes formed with mutant antigen A can be removed from the sample by techniques known to those skilled in the art prior to bringing the sample into contact with native antigen A. This will help to eliminate any unspecific binding from this essay.

In one embodiment of the present invention the method comprises the following steps:

a) Obtaining a first sample suspected of containing anti-A-antibodies from a subject to be analysed,
b) Providing the native antigen A,
c) Contacting the first sample suspected of containing anti-A-antibodies with the native antigen A,
d) Detecting the amount of bound anti-A-antibodies after step c),
e) Providing mutant antigen A,
f) Obtaining a second sample suspected of containing anti-A-antibodies from the same subject to be analysed as in step a),
g) Contacting the second sample suspected of containing anti-A-antibodies from the same subject as in step a) with mutant antigen A,
h) Detecting the amount of bound anti-A-antibodies after step g),
i) Determining the ratio and/or the difference of anti-A-antibodies bound to antigen A of step d) to anti-A-antibodies bound to mutant antigen A of step h),
wherein the ratio and/or the difference of anti-A-antibodies bound to antigen A compared to anti-A-antibodies bound to mutant antigen A allows the diagnosis, the categorisation, the prediction and/or the monitoring of the progression of a condition.

For the purpose of the present invention is a sample suspected of containing anti-A-antibodies any sample that is obtained in order to check it for anti-A-antibodies. Thus, for a sample to be suspected of containing anti-A-antibodies it is not necessary, that there is reason to believe that the sample might contain anti-A-antibodies, in particular it is not necessary that symptoms for the condition associated with anti-A-antibodies already show.

The sample suspected of containing anti-A-antibodies can be in principle any sample obtained from an organism that contains antibodies. It is preferred, that the first and the second sample are derived from the same origin in the subject to be analysed, e.g., both are blood samples.

It is even more preferred that only one sample is obtained from the subject to be analysed that after removal is split into two portions, one of which then serves as first sample and the other one serves as second sample, in order to ensure sample homogeneity.

As sample size for carrying out the method of the present invention, 1-5 preferably 1-25 μl, even more preferred 1-1000 μl is sufficient, although larger samples are usable, too. Using equal sample volumes for the first and the second sample is preferred, because equal amounts of both samples will simplify the comparison of anti-A-antibodies bound to antigen A and anti-A-antibodies bound to mutant antigen A.

Preferably, the samples employed as first and second sample should have an anti-A antibody concentration of about 1 μg/ml-0.001 μg/ml, in particular preferred of 0.5 μg/ml-0.01 μg/ml.

Undiluted samples, as they are obtained from a subject to be analysed, e.g., from a human, should have a total antibody A concentration of at least 1 μg/ml, more preferred 10 to 100 μg/ml, even more preferred 10 μg/ml to 1 mg/ml or even higher if available.

Prior to the analysis with the method of the present invention the samples are preferably diluted to a desired total antibody A concentration of, e.g., 1 μg/ml to 0.1 μg/ml.

Antigen A and mutant antigen A are preferably provided in equal molar amounts. The total amount of antigen A and mutant antigen A used in each experiment is 0.1-100 μg, preferably 0.2-50 even more preferred 0.3-25 μg, most preferred 0.5-10 μg. More antigen can be provided, however this will require rather large amounts of protein.

It is one advantage of the method of the present invention, that it is possible to surprisingly improve the accuracy of the analysis methods of the state of the art, while still requiring extremely small sample volumes.

To bring the first sample suspected of containing anti-A-antibodies in contact with the native antigen A, any method is suitable that allows an antigen-antibody-interaction to take place.

Similarly, to bring the second sample suspected of containing a anti-A-antibodies from the same subject as in step a) in contact with mutant antigen A, any method is suitable that allows an antigen-antibody-interaction to take place.

It is preferred, even though not required, that the sample suspected of containing anti-A-antibodies is brought into contact with antigen A by the same method as the second sample is brought into contact with mutant antigen A.

After formation of antigen-antibody interaction any method can be used to detect and quantify the formed antigen-antibody-complexes that can discriminate antigen-antibody-complexes from the remaining components of the samples. Quantitative chromatography such as gel chromatography, column chromatography, in particular size exclusion chromatography, chromatography based on ionic interactions or affinity chromatography, density centrifugation or simple filtering are only some examples of applicable methods. Other alternatives are optical methods, such as electron microscopy or light scattering. Those skilled in the art will know, how these methods are carried out and how they can be used to quantify the components of a sample.

Those skilled in the art will also be able to use alternative methods that are known in the art to quantify antigen-antibody-complexes.

In one embodiment of the present invention the determination of the ratio and/or the difference of anti-A-antibodies bound to antigen A of step d) compared to anti-A-antibodies bound to mutant antigen A of step h) is simply carried out by calculation by hand.

In a preferred embodiment of the present invention, the amounts of anti-A-antibodies bound to antigen A of step d) and of anti-A-antibodies bound to mutant antigen A of step h) are measured by a detection means, which then transfers corresponding signals to a computational unit. The computational unit will then calculate the ratio and/or the difference of anti-A-antibodies bound to antigen A of step d) compared to anti-A-antibodies bound to mutant antigen A of step h) and transmit a corresponding signal to a display unit which displays the obtained ratio and/or the difference.

In one embodiment of the present invention the method of the present invention further comprises the step of providing the antigen A and/or the mutant antigen A with at least one detectable moiety.

A detectable moiety is any atom or group of atoms that alone or after activation, possibly after combination with another reagent, emits a signal. This signal can be emitted permanently or only after binding to the antibody or until the antigen provided with the detectable moiety is bound to a corresponding antibody. In case the detectable moiety emits a signal only after activation, it is possible to first remove all unbound antigens with a detectable moiety from the sample and then to activate the detectable moiety.

If antigen A and mutant antigen A are provided with a detectable moiety it is possible to provide both antigens with the same detectable moiety or with different detectable moieties.

Providing both antigens with the same detectable moiety has the advantage, that in the quantification step the obtained signals are easy to compare and errors from different detection systems for different signals are avoided.

Providing both antigens with different detectable moieties has the advantage that in this case it is possible to carry out the invention in a one-pot assay. Antigen A provided with a first detectable moiety and mutant antigen A provided with a detectable moiety that is different from the first detectable moiety are in this case brought into contact simultaneously with the sample suspected of containing anti-A-antibodies. The ratio and/or the difference of anti-A-antibodies bound to antigen A of step d) compared to anti-A-antibodies bound to mutant antigen A of step h) is then obtained as ratio and/or the difference of the signal of the detectable moiety of antigen A compared to the signal of the detectable moiety of mutant antigen A.

If only one of antigen A or mutant antigen A is provided with a detectable moiety, then it is again possible to carry the method of the present invention out as a one-pot-reaction. In case antigen A is provided with a detectable moiety and mutant antigen A is not provided with a detectable moiety, equal amounts of antigen A and mutant antigen A are brought into contact simultaneously with the sample suspected of containing anti-A-antibodies. As reference sample, an equal amount of the sample suspected of containing anti-A-antibodies is simultaneously brought into contact with antigen A labelled with a detectable moiety in similar amounts as it is present in the mixture of antigen A and mutant antigen A. The ratio and/or the difference of anti-A-antibodies bound to antigen A of step d) to anti-A-antibodies bound to mutant antigen A of step h) is then obtained from the difference of the signals of the mixed and of the reference sample.

The detectable moiety is preferably selected from the group consisting of radioactive markers or enzymes, such as, e.g., alkaline phosphatase or horseradish peroxidase, colloidal gold, urease, fluorescein, rhodamine, and biotin-streptavidin.

According to one embodiment of the present invention the individual steps of the described method are carried out in the framework of an immuno-absorbance essay, in particular in an enzyme-linked immunosorbent assay (ELISA), radioimmunoassay (RIA), BIACORE or an enzyme immuno assay (EIA), preferably in an automated form.

These assays are state of the art and those skilled in the art will know, how to use the method and the kit of the present invention in these analysis methods.

RIA is a method used to test antigens without the need to use a bioassay. It involves mixing known quantities of a radioactively labelled antigen, frequently labelled with radioactive isotopes of iodine attached to tyrosine, with antibodies specific to that antigen, then adding unlabeled or “cold” antigen and measuring the amount of labelled antigen displaced.

The Biacore technology is based on the natural phenomenon of surface plasmon resonance. A protein, e.g. an antigen, is attached to the sensor surface, while the ligand, e.g. a specific antibody, is part of the mobile phase which is running along the surface. On the backside of the sensor surface light is reflected with an intensity that changes when the ligand from the mobile phase binds to the fixed protein.

EIA is an assay that uses enzyme-bound antibodies to detect antigens or enzyme bound antigens to detect antibodies. The enzyme catalyses a reaction with a detectable product when exposed to a substrate.

The method of the present invention is ideally suited to be carried out in an automated form. For example, antigen A and the mutant antigen A, both labelled with a detectable moiety can be added into a multiwell-plate. A multitude of samples suspected of containing anti-A-antibodies can then be added thereto, the formed antibody-antigen complexes can be automatically detected thereafter and the desired ratios and differences can be calculated by a computer. This would allow to screen a large number of patients simultaneously for a particular condition and/or disorder.

Similarly, a single sample of a subject to be tested can be brought into contact with a multitude of antigens and corresponding mutant antigens. This way, an individual can be tested simultaneously for a multitude of conditions, for example for research purposes or as part of a medical check-up.

Variations of these automated methods according to the present invention can be made by and are within the skill of those skilled in the art and are part of the present invention.

In the present invention it is preferred that the condition to be diagnosed, to be categorised and/or its progression to be monitored is a physiological or a clinical condition.

In particular, the subject matter of the present invention can be used to diagnose and/or categorise cancers, in particular carcinoma, lymphoma, leukaemia, sarcoma, mesothelioma, gliome, germ cell tumors and choriocarcinoma and/or to predict and/or monitor their progression.

The subject matter of the present invention can also be used to diagnose and/or to categorise an infectious disease. An infectious disease in this respect is a disease caused by a biological agent such as, e.g., a virus, a bacterium, a fungi and protozoa, or a parasite. Examples of infectious diseases that can be diagnosed, categorised, predicted and/or their progression monitored are lower respiratory infections, HIV/AIDS, diarrhea) diseases, tuberculosis (TB), malaria, measles, pertussis, tetanus, meningitis, syphilis, hepatitis B, poliomyelitis, diphtheria and tropical diseases, such as, e.g., chagas disease, dengue fever, lymphatic filariasis, leishmaniasis, onchocerciasis, schistosomiasis and trypanosomiasis.

One important application of the subject matter of the present invention is to check the success of a vaccination and to monitor the status of a vaccination. In particular in disease control programs the subject matter of the present invention can be applied to check the status of vaccination of whole populations. In particular the applicability of the subject matter of the present invention to automated analysis methods, in particular to high throughput screening is very useful in this respect.

In one further embodiment the subject matter of the present invention is used to diagnose, to categorise, to predict and/or to monitor the progression of an auto immune disorder such as, e.g., Hashimoto's thyroiditis, pernicious anemia, Addison's disease, diabetes, in particular type I, rheumatoid arthritis, systemic lupus erythematosus, dermatomyositis, Sjogren's syndrome, lupus erythematosus, myasthenia gravis, Reiter's syndrome and Grave's disease.

In particular the subject matter of the present invention can be used to diagnose, to categorise, to predict and/or to monitor the progression of EAE and/or MS.

The ratio or the difference of anti-A-antibodies bound to antigen A compared to anti-A-antibodies bound to mutant antigen A, that allows the diagnosis, the categorisation, the prediction and/or the monitoring of the progression of a condition can be obtained from reference examples obtained from individuals that exhibit the particular condition.

Contrary to the diagnosis methods of the prior art, the subject matter of the present invention surprisingly overcomes the problems that arise from the general incomparability of samples of different individuals because of factors such as race, sex, area of living, lifestyle, age, previous antigens encountered, inheritance, other present diseases or nutrition.

Hence, the measured difference and/or ratio of one individual that suffers from a condition can serve as a reference example and provide indicative figures that allow the diagnosis of the same condition in other individuals.

Based thereon it is possible to establish a meaningful databank with reference figures that allow the diagnosis, the categorisation, the prediction and/or the monitoring of the progression of different conditions.

In general, the medical practitioner will know, what ratio and/or what difference is indicative for a certain condition.

Usually, a ratio of anti-A-antibodies bound to antigen A to anti-A-antibodies bound to mutant antigen A of >1, preferably of >1.5, in particular preferred of >2 allows the diagnosis of a particular condition.

In one embodiment of the present invention the sample suspected of containing anti-A-antibodies from a subject is immobilised on a matrix prior to the contact with the antigen A and/or mutant antigen A. This has the advantage that after contact with the antigen A and the mutant antigen A the formed antigen-antibody complexes will remain bound on the matrix, whereas any unbound antigen A or mutant antigen A can be washed off from the matrix. Thereafter a readout of a detectable signal can be obtained directly from the matrix with the bound antigen-antibody complexes thereon.

It is furthermore possible to immobilise the native antigen A and/or the mutant antigen A on a matrix prior to the contact with the sample suspected of containing anti-A-antibodies from a subject. This has the advantage that after contact with anti-A-antibodies only antigen-anti A antibody-complexes will remain bound on the matrix, whereas the remaining components of the sample can be washed off from the matrix, so that the possibility that they might interfere with the measured signal is eliminated.

Finally, it is also possible to immobilise both, the antigens and the antibodies on a matrix, as long as it is still possible for the antigens and antibodies to interact. Also this approach has the advantage, that other components of the sample can be easily removed before a signal is measured.

Washing is an optional step after contacting antigen A or mutant antigen A with the anti-A-antibody in the subject matter of the present invention. Washing can help to remove any sample components from the sample that might interfere with the generation or detection of a detectable signal.

Washing in this respect can be carried out with polar solvents, in particular aprotic solvents such as, e.g., 1,4-Dioxane, tetrahydrofuran (THF), acetone, acetonitrile (MeCN), dimethylformamide (DMF), dimethyl sulfoxide (DMSO) or protic solvents such as, e.g., acetic acid, n-butanol, isopropanol, n-propanol, ethanol, methanol, formic acid, water or mixtures thereof.

It is preferably, that the solvents are buffered at a pH, that can be tolerated by the antibody-antigen-complexes, such as, e.g., pH 2-11, 3-10, 4-9, 5-8, particularly preferred pH 6.5-7.5, and mostly preferred pH 7.3

Suitable buffers are any buffers that buffer at these pH-ranges. Preferred are, e.g., TAPS (tris(hydroxymethyl)methyl]amino}propanesulfonic acid), bicine (N,N-bis(2-hydroxyethyl)glycine), tris (tris(hydroxymethyl)methylamine), tricine (N-tris(hydroxymethyl)methylglycine), HEPES (4-2-hydroxyethyl-1-piperazineethanesulfonic acid), TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), Cacodylate (dimethyl arsenate), MES (2-(N-morpholino)ethanesulfonic acid) and/or acetate, PBS (phosphate buffered saline).

In one embodiment of the present invention the method further comprises the step of contacting the anti-A-antibody-antigen A complexes after step c) and/or the step of contacting the anti-A-antibody-mutant antigen A complexes after step g) with a secondary antibody binding antibody.

In another embodiment the method of the present invention further comprises the step of contacting the anti-A-antibody-antigen A complexes after step c) and/or the step of contacting the anti-A-antibody-mutant antigen A complexes after step g) with a secondary antigen A-binding antibody.

According to further embodiments of the present invention the secondary antibody binding antibody and/or the secondary antigen A-binding antibody contains a detectable moiety. As with respect to the detectable moiety that the antigens can be provided with, the detectable moiety for the secondary antibodies can also be any atom or group of atoms that alone or after activation, possibly after combination with another reagent emits a signal and is preferably selected from the group consisting of radioactive markers, enzymes, such as, e.g., alkaline phosphatase or horseradish peroxidase, colloidal gold, urease, fluorescein, rhodamine, and biotin-streptavidin.

In this respect and as mentioned above, the subject matter of the present invention is ideally suited to be used in the framework of an ELISA assay.

ELISA uses at least one antibody that is specific to the antigen and another so-called secondary antibody that can be provided with a detectable moiety, such as an enzyme, e.g., alkaline phosphatase or horseradish peroxidase.

This secondary antibody, e.g. provided with alkaline phosphatase or horseradish peroxidase as detectable moiety can cause, e.g., a chromogenic and/or fluorogenic substrate to produce a signal.

ELISA can be performed to evaluate the presence of anti-A-antibodies in a sample, it is thus a useful tool for determining serum antibody concentrations for one or more conditions to be investigated.

The steps of ELISA for determining the presence of anti-A-antibodies and or their concentrations can be for example:
Applying a sample of antigen A to a surface, often the well of a microtiter plate. The antigen can be fixed to the surface to render it immobile.
Washing the plate to remove unbound antigen.
Applying a large amount of an unreactive agent (blocking agent) to the surface that does not or does hardly bind antibodies (e.g. bovine serum albumin) to bind to empty spaces that are not occupied by the antigen A.
Washing the plate to remove unbound blocking agent.
Applying samples suspected of containing anti-A-antibodies of unknown antibody concentration, usually in a diluted form, to the plate. Additional reagents like bovine serum albumin can be added to the solution to stabilize the antibodies and to reduce unspecific binding.
Washing the plate, so that any unbound antibodies are removed. After this wash, only the anti-A-antibody-antigen A complexes remain attached to the well.
Adding the secondary antibodies to the wells, which will bind to any antigen-antibody complexes. These secondary antibodies are, e.g., provided with an enzyme, that is capable of producing a signal, once it can interact with a substrate.
Washing the plate, so that excess unbound secondary antibodies are removed.
Applying a substrate which is converted by the enzyme to elicit a detectable signal.
Detecting the signal.
Repeating the procedure with mutant antigen A instead of antigen A.
Determining the ratio and/or the difference of anti-A-antibodies bound to antigen A compared to anti-A-antibodies bound to mutant antigen A from the detected signals.

In this method the enzyme can act as an amplifier: even if only few enzyme-linked antibodies remain bound, the enzyme molecules will produce many signal molecules. To evaluate the obtained optical density or fluorescent units of the sample advantageously a standard curve can be used for interpolation, that can be obtained from a set of experiments using a serial dilution of the secondary antibody provided with the enzyme and/or of the substrate.

An alternative for an applicable ELISA-method is a “double antibody sandwich ELISA” technique.

The steps are, e.g., as follows:
Binding an antibody to the wells of the plate that specifically binds antibodies of the species from which the anti-A antibodies are obtained.
Washing the plate, so that any unbound antibody is removed.
Applying a large amount of an unreactive agent (blocking agent) to the surface that does not or hardly bind antibodies (e.g. bovine serum albumin) to bind to empty spaces that are not occupied by the antibody.
Washing the plate to remove unbound blocking agent.
Applying samples suspected of containing anti-A-antibodies of unknown antibody concentration, usually in a diluted form, to the plate.
Washing the plate, so that any unbound components are removed.
Applying antigen A to the plate that is specifically bound by anti-A antibodies.
Washing the plate to remove unbound antigen A.
Applying secondary enzyme-linked antibodies to the plate which are also specific to the antigen A, however that bind at a position that differs from the position that the anti-A-antibodies bind to.
Washing the plate, so that unbound enzyme-linked antibodies are removed.
Applying a substrate which is converted by the enzyme into a detectable signal.
Detecting the signal and quantifying it.
Repeating the procedure with mutant antigen A
Determining the ratio and/or the difference of anti-A-antibodies bound to antigen A compared to anti-A-antibodies bound to mutant antigen A from the detected signals.)

A third possible alternative for an applicable ELISA-method is a variant of the “competitive ELISA” technique.

The steps for this ELISA method can be, e.g., as follows:
The sample suspected of containing anti-A-antibodies is incubated in the presence antigen A to form antibody-antigen complexes.
This sample comprising the bound antibody/antigen complexes is then added to an antigen A coated well.
The plate is washed, so that any unbound antibody is removed. The more anti-A-antibodies were present in the sample, the more anti-A-antibodies will still available for binding to the immobilised antigen A in the well, hence “competition”.
The secondary antibody, specific to the primary anti-A-antibody is added. This secondary antibody is coupled to an enzyme.
A washing step is employed to remove all unbound secondary antibodies.
A substrate of the enzyme is applied, which is converted by the enzyme into a detectable signal, preferably a chromogenic or fluorescent signal.
The signal is detected and quantified.
The procedure is repeated with mutant antigen A
The ratio and/or the difference of anti-A-antibodies bound to antigen A compared to anti-A-antibodies bound to mutant antigen A is determined from the detected signals.

Possible matrices used in the present invention to immobilise antigen A and/or mutant antigen A and/or anti-A-antibodies can be any material that antigen A and/or mutant antigen A and/or anti-A-antibodies can be attached to without disabling the antigen-binding capacity of the antibodies or the antibody-binding capacity of the antigens. Preferably is the matrix a membrane, a cell membrane, a chip, a dish, an ELISA well, a tube, in particular a plastic or a glass tube, a cuvette, a polymer particle, a bead, a pellet or a resin for a chromatographic column.

The sample used in the framework of the present invention can be any sample that potentially contains antigens, in particular antigen A. It is, however, preferred that the sample is a blood sample, a cerebrospinal fluid sample, a CNS sample or a serum sample of a patient.

The amount of bound antibodies can be detected depending on the kind of detectable moiety used, if any. If a detectable moiety is used, it is within the skill of those skilled in the art to select a suitable method of detection. Preferably the generated signals are detected by visual or automated detection, e.g., by spectrometry, preferably of a precipitate or a colour change, by light or electron microscopy, by radiometric measurements or by fluorescence microscopy.

It is wherever appropriate preferred, to calibrate the method of detection and/or the employed detection means, e.g., by using a dilution series of antibodies provided with enzymes as detectable moieties and corresponding substrates. The calibration of such a detection method is within the skill of a person skilled in the art.

The subject matter of the present invention is applicable independently of the nature of the antigen. Any antigen, such as, e.g., foreign proteins, viruses, fungi, bacteria, and also substances such as toxins, chemicals, drugs, and other particles that are foreign to an organism, can be used as native antigen A. Preferably, the native antigen A is selected from the group consisting of Ro, La, Jo-1, SM, Scl70, SS-A, SS-B, Pr3, MPO, thyroglobulin, TPO, thyrotropin receptor, insulin, insulin receptor, GAD, DNA topoisomerase II, IA-2, IA-2beta, TSH receptor, PM/Scl100, acetyl choline receptor, BP180, NC1, Histone, U1 RNP, tissue transglutaminase, type IV collagen, MOG and MBP. All these antigens are known in the art (Mahler, M., Bluthner, M. & Pollard, K. M. (2003) Clinical Immunology 107, 65-79; Scofield, R. H. (2004) Lancet 363, 1544-1546; D′Cruz, D. (2002) Toxicology Letters 127, 93-100; and references therein). Additionally, the employed antigen A can also comprise only antigenic domains of antigens or can comprise antigenic parts of these antigens that share an amino acid sequence homology with the complete native antigen sequence of at least 10% identical amino acids, preferably at least 25% identical amino acids, more preferred at least 50% identical amino acids and in particular preferred at least 75% identical amino acids.

In general the native antigen A can be obtained by any method known in the art. It is preferred, however, that the antigen A and/or the mutant antigen A is provided from a recombinant expression system. If the sequence of an antigen is known, it is within the skill of those skilled in the art to select a suited expression system, in particular an appropriate vector and an appropriate organism along with appropriate growth conditions for protein expression.

Using recombinant protein expression has the advantage that it is possible to generate large amounts of protein in a short period of time with relatively inexpensive equipment and at low costs.

Oftentimes, expression systems work so well that quantities of protein are generated that are no longer folded correctly but that are expressed in inclusion bodies instead. Inclusion bodies contain denatured protein.

Denatured protein is in general much easier to handle and to store than protein in its native fold. Denatured antigen A can be transformed into its native state by a procedure called “refolding”. It is within the skill of those of skill in the art to select proper refolding conditions for a particular denatured antigen.

According to one embodiment of the present invention the native antigen A and/or the mutant antigen A is used in a refolded form.

The mutant antigen A used in the subject matter of the present invention comprises at least one altered amino acid with respect to the native antigen A sequence that is located within an epitope of the native antigen A.

In particular, the mutant antigen A used in the subject matter of the present invention comprises 1, 2, 3, 4, 5, 6, 7, 8, 9 or 10 altered amino acids with respect to the native antigen A sequence that are located within an epitope of the native antigen A.

An epitope is the part of a molecule that is recognised by the immune system, specifically B-cell epitopes are recognized by antibodies or B cells and T-cell epitopes by T-cells, or T cells. In the following “epitope” stands for B-cell epitope. It is within the skill of those skilled in the art to determine such epitopes; in particular they can be mapped by techniques such as using protein microarrays, ELISPOT or ELISA.

Most epitopes that are recognised by antibodies and B-cells can be thought of as three-dimensional surface features of an antigen molecule; that fit precisely and thus bind to the anti-A-antibody, in particular to its paratope. Exceptions are linear epitopes, which are determined by the amino acid sequence, the primary structure, rather than by the tertiary structure of a protein.

In one embodiment of the present invention the native antigen A is Myelin Oligodendrocyte Glycoprotein (MOG) or comprises antigenic parts of MOG that share an amino acid sequence homology with the native MOG sequence of at least 10 identical amino acids, preferably at least 25 identical amino acids, more preferred at least identical 50 amino acids and in particular preferred at least 75 identical amino acids.

The present inventors were able to solve the three dimensional protein structure of MOG (Breithaupt et al., 2003, Proceedings of the National Academy of Sciences of the United States of America, 100: 9446-51). Using this structure, it was possible to define amino acids that are located on the surface of MOG and that, hence can contribute to the formation of epitopes.

Consequently, the mutant antigen A is MOG or an antigenic part of MOG that shares an amino acid sequence homology with the native MOG sequence of at least 10 identical amino acids, preferably of at least 25 identical amino acids, more preferred of at least identical 50 amino acids and in particular preferred of at least 75 identical amino acids where at least one amino acid is altered with respect to the native MOG sequence, preferably is the at least one altered amino acid located within the MOG-sequence that is part of an epitope, more preferred of the immuno dominant epitope, even more preferred is the at least one altered amino acid located within amino acids 28-35, 42-55, 72-80, 86-93 and/or 101-108 of the native MOG sequence, still more preferred within the FG-loop of native MOG, namely the amino acids 101-108, preferably contains the mutant MOG-sequence 1, 2, 3, 4, 5, 6, 7 or 8 mutations, in particular preferred is the mutant antigen A selected from the group consisting of the single mutant Ser104Glu, the double mutant His103Gly, Ser104Glu and the double mutant His103Ala, Ser104Glu.

The present invention also comprises an assay wherein the mutant antigen A (e.g. mutant MOG) is used to bind (absorb) all molecules (e.g. unspecific binding antibodies) that are present in the sample of interest and that contribute to the background of the assay when it is used to determine the amount of specific antibodies against the particular antigen A (e.g. MOG). Two variations of the method can be applied:

1. The mutant antigen A (e.g. MOG-mutant) is added directly to the sample to be measured. The advantage would be for example in an ELISA assay that contains pre-bound antigen (e.g. MOG) that substances (e.g. unspecific antibodies) that would bind to the antigen unspecifically and that would contribute to the background of the assay will also bind to the added but soluble mutant antigen (or artificial polymers of the mutant antigen). In the following washing steps these unspecific binders can be washed away prior to the detection step.
2. Alternatively the sample can be depleted from substances that react unspecifically with antigen A (e.g. MOG) by incubating the sample with a material (e.g. chromatography raisin) to which the mutant antigen A (e.g. mutated MOG) is attached. In this case the unspecific binders remain bound to the raisin and are removed from the sample of interest.

The result of both procedures is an increase of the signal to noise ratio when the sample is tested for specific antibodies against antigen A.

The subject matter of the present invention is in general applicable to any organism that exhibits an immune system. The present inventors, however, intend to use the subject matter of the present invention primarily for mammalian subjects, in particular humans.

Also comprised by the subject matter of the present invention is a kit for carrying out the method of the present invention comprising a native antigen A and a mutant antigen A.

Preferably, the kit of the present invention is a kit to diagnose, to categorise, to predict and/or to monitor the progression of EAE and/or MS comprising

a) native MOG or antigenic parts of MOG that share an amino acid sequence homology with the native MOG sequence of at least 10 identical amino acids, preferably of at least 25 identical amino acids, more preferred of at least identical 50 amino acids and in particular preferred of at least 75 identical amino acids;
b) mutant MOG or an antigenic part of MOG that shares an amino acid sequence homology with the native MOG sequence of at least 10 identical amino acids, preferably of at least 25 identical amino acids, more preferred of at least identical 50 amino acids and in particular preferred of at least 75 identical amino acids; where at least one amino acid is altered with respect to the native MOG sequence, preferably is the at least one altered amino acid located within the MOG-sequence that is part of an epitope, more preferred of the immuno dominant epitope, even more preferred is the at least one altered amino acid located within amino acids 28-35, 42-55, 72-80, 86-93 and/or 101-108 of the native MOG sequence, still more preferred within the FG-loop of native MOG, namely the amino acids 101-108, preferably contains the mutant MOG-sequence 1, 2, 3, 4, 5, 6, 7 or 8 mutations, in particular preferred is the mutant antigen A selected from the group consisting of the single mutant Ser104Glu, the double mutant His103Gly, Ser104Glu and the double mutant His 103Ala, Ser104Glu.

In one embodiment, the kit of the present invention can also comprise a secondary antibody-binding antibody and/or a secondary MOG binding antibody.

Furthermore, the kit of the present invention can comprise a detectable unit linked or to be linked to the native MOG and/or mutant MOG and/or secondary antibody-binding antibody and/or secondary MOG binding antibody, preferably a radioactive marker, an enzyme such as, e.g., alkaline phosphatase or horseradish peroxidase, colloidal gold, urease, fluorescein, rhodamine, biotin-streptavidin.

According to one embodiment of the present invention the kit also comprises a matrix to immobilise the antigens and/or the antibodies wherein the matrix is preferably a membrane, a cell membrane, a polymer particle, a chip, a dish, an ELISA well, a tube, in particular a plastic or a glass tube, a cuvette, a bead, a pellet or a resin for a chromatographic column.

On embodiment of the present invention comprises a chip or an ELISA well provided with an array of different antigens and mutant antigens immobilised thereon. Such a chip or ELISA well can be used to screen for multiple conditions simultaneously and would be ideally suited for automated applications.

In the kit of the present invention at least one of the antigens or antibodies can be provided in a lyophilised or denatured form. This would allow an easier handling, a prolonged storage time and a longer lifetime of the kit. In this case it is preferred that the kit further comprises a corresponding refolding solution that allows to refold the antigens or antibodies prior to their use.

Finally, the kit of the present invention can furthermore comprise a washing solution, preferably a polar washing solution, in particular preferred buffered water.

Washing solutions can be any polar solvents, in particular aprotic solvents such as, e.g., 1,4-Dioxane, tetrahydrofuran (THF), acetone, acetonitrile (MeCN), dimethylformamide (DMF), dimethyl sulfoxide (DMSO) or protic solvents such as, e.g., acetic acid, n-butanol, isopropanol, n-propanol, ethanol, methanol, formic acid, water or mixtures thereof. Preferred is buffered water. It is preferred, that the solvents are buffered at a pH, that can be tolerated by the antibody-antigen-complexes, such as, e.g., pH 2-11, 3-10, 4-9, 5-8, and particularly preferred pH 6.5-7.5. Suitable buffers are any buffers that buffer at these pH-ranges. Preferred are, e.g., TAPS (tris(hydroxymethyl)methyl]amino}propanesulfonic acid), bicine (N,N-bis(2-hydroxyethyl)glycine), tris (tris(hydroxymethyl)methylamine), tricine (N-tris(hydroxymethyl)methylglycine), HEPES (4-2-hydroxyethyl-1-piperazineethanesulfonic acid), TES (2-{[tris(hydroxymethyl)methyl]amino]ethanesulfonic acid), MOPS (3-(N-morpholino)propanesulfonic acid), PIPES (piperazine-N,N′-bis(2-ethanesulfonic acid)), Cacodylate (dimethyl arsenate), MES (2-(N-morpholino)ethanesulfonic acid) and/or acetate, PBS (phosphate buffered saline).

Further features and advantages of the subject matter of the present invention will be apparent from the following examples and drawings:

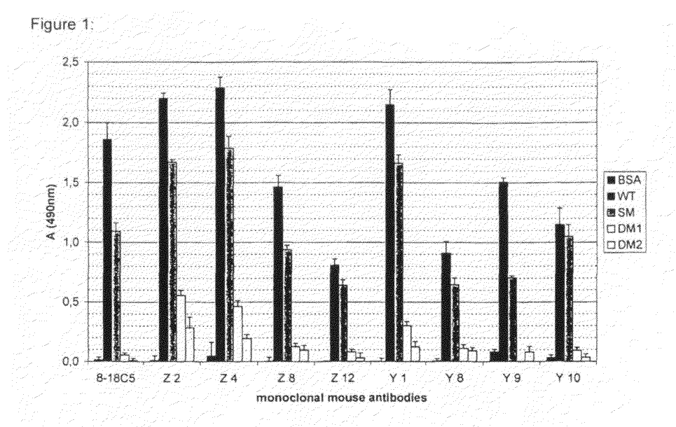

FIG. 1 shows the extend of monoclonal mouse anti-MOG antibody—binding to rat—MOG (WT), rat MOG mutants (SM, S42P, DM1, DM2), human MOG (hMOG) and BSA as control. The data were obtained according to the procedure described in example 1. It is evident from FIG. 1 that antibody binding to the MOG mutants SM, DM1 and DM2 that contain mutations in the antigenic FG loop is strongly reduced for all monoclonal antibodies. The double mutants DM1 and DM2 yield an ELISA signal of 0 to 25% compared to the not mutated MOG (WT). The single mutant SM yields ELISA signals in the range of 47% to 79%.

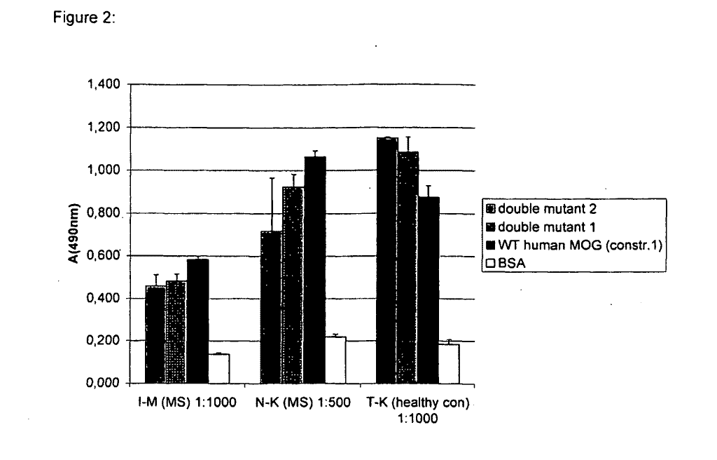

FIG. 2 shows the result of an experiment described in detail in example 2. Sera were obtained from healthy individuals and from MS patients. These serum samples were brought into contact with human MOG (WT) and two mutant rat MOGs and with BSA as control. Displayed is the amount of antibody binding to the presented antigens.

### EXAMPLE 1

### Binding of Several Mouse Monoclonal Antibodies to MOG and its Mutants

### Design of Mutant MOG and Site-Directed Mutagenesis

The protein crystal structure of the extracellular domain of MOG (MOGex) was recently solved (Breithaupt et al., 2003, Proceedings of the National Academy of Sciences of the United States of America, 100: 9446-51). Based on this structure possible intermolecular contacts between MOG as antigen and corresponding antibodies were analyzed using programs of the program package CCP4 (Collaborative Computational Project, 1994, Acta Crystallographica Section D-Biological Crystallography, 50:760-763.) and the model building program O (Jones et al., 1991, Acta Crystallographica Section a, 47:110-119.). Electrostatic potentials were calculated in GRASP (Nicholls et al., 1991, Proteins-Structure Function and Genetics, 11:281-296) by employing atomic charges according to Weiner and colleagues (Weiner et al., 1984; Journal of the American Chemical Society, 106(3), 765-784). The solvent accessible surface of MOGₑₓ was calculated with the utility SURFACE of the CCP4 program package.

Mutagenesis was carried out using the extracellular domain of rat MOG (MOGₑₓ) subcloned into the His-tag expression vector pQE-12 by following the method of “QuikChange Site-Directed Mutagenesis” by Stratagene (LaJolla, USA). The oligonucleotides used were: 5′-CTTCAGAGA CCACGAATA CCAAGAAGA AGCCGCCG-3′ (SM1, Ser104Glu), 5′-CACATGCTT CTTCAGAGA CGGCGAATA CCAAG-3′ (DM1, His103Gly, Ser104Glu), 5′-CACATGCTT CTTCAGAGA CGCTGAATA CCAAG-3′ (DM2, His103Ala, Ser104Glu) and the corresponding reverse complementary oligonucleotides. The identity of the mutations was verified by DNA sequencing of the purified plasmids.

### Protein Expression and Refolding of Recombinant MOG

Plasmids containing the extracellular domain of human MOG and the “humanized” rat MOG mutant Ser42Pro were a kind gift of Nancy Ruddle (Oliver et al., 2003, Journal of Immunology 171(1), 462-468). The extracellular domain of rat and human MOG and the mutant proteins were overexpressed in inclusion bodies in Escherichia coli. After disruption of the cells by sonification the inclusion bodies were purified by repetitive steps of centrifugation and resuspension in 50 mM Tris/HCl (pH 8.0), 0.3 M NaCl, 0.5% LDAO. The inclusion bodies were solubilized in solubilisation buffer (100 mM NaH₂PO₄, 10 mM Tris, 6 M guanidinium chloride, 40 mM mercaptoethanol, pH 8.0). After dilution in mercaptoethanol-free solubilisation buffer the denatured MOG was bound to Ni-NTA Superflow (Qiagen, Hilden, Germany) material and refolded on the column in two steps. At first, a linear gradient from solubilisation buffer (1 mM mercaptoethanol) to 100 mM NaH₂PO₄, 10 mM Tris, 3 mM glutathione, pH 8.0 over 10 hours and 80 column volumes was applied, followed by a short linear gradient (2 hours, 2 column volumes) to remove the glutathione for complete oxidation of the refolded MOG. After elution, unfolded and aggregated MOG was removed by a final gel filtration chromatography step. Identity and integrity of the proteins were checked by mass spectrometry and one-dimensional ¹H-NMR. Protein concentrations were determined by UVN is spectroscopy, relative concentrations by the Bradford protein assay (BioRad, Hercules, USA).

### ELISA

Antibody binding to MOG and to the mutant proteins was measured by ELISA. The mouse monoclonal antibodies (mAb) 8-18C5 (Linnington et al., 1984; Journal of Neuroimmunology, 6:387-96.), Y1, Y8, Y9, Y10, Z2, Z4, Z8 and Z12 (Piddlesden et al., 1993, American Journal of Pathology, 143:555-564) were purified from hybridoma supernatants by affinity chromatrography on Protein G. Their concentration was estimated by UVN is spectroscopy and colorimetrically by the Bradford method. 96-Well plates (Maxisorb, Nunc, Rosklide, Denmark) were coated with 100 μL 10 μg/ml antigen in PBS (1 h, 30° C.), washed three times with PBS containing 0.2% Tween20 and blocked with PBS containing 1% w/v BSA (2 h, 30° C.). After washing, the plates were incubated with the monoclonal antibodies (˜0.5 μg/ml in PBS) or the plasma samples of the MOG-vaccinated mice diluted 1:250 for 1 h at 30° C. The washing procedure was repeated and anti-mouse IgG (Fab′)₂, conjugated with horseradish peroxidase (Amersham Biosciences, Uppsala, Sweden), that was diluted 1:10000 in PBS was added and the plates were incubated for 1 h at 30° C. Antibody binding was detected by oxidation of o-phenylene diamine and quantified by measuring the absorbance at 490 nm after stopping the reaction with H₂SO₄. The in FIG. 1 displayed values correspond to the means of triplicate (plasma samples) and quadruplicate (hybridoma supernatants) measurements of a representative experiment.

### Example 2

### Binding of Human Antibodies Obtained from Patients Suffering from MS and from Healthy Controls to Native MOG and to Two Mutant MOGs

Samples of human sera obtained from two patients suffering from MS, I. M. and N. K. and one serum sample obtained from T. K. as healthy control were brought into contact with wildtype human MOG and with double mutant rat MOG (double mutant 1, His103Gly, Ser104Glu; and double mutant 2, His103Ala, Ser104Glu) by using the following protocol:

(1) Coat 96 well ELISA plates with 100 μl 10 μg/ml MOG, MOG mutants and BSA (control).
(2) Remove unbound antigen by washing 3× with 240 μl PBS/0.2% Tween 20.
(3) Block the plates with 240 μl 2% BSA dissolved in PBS/0.02% sodium azide.
(4) Remove unbound BSA by washing 3× with 240 μl PBS/0.2% Tween 20.
(5) Incubate plates with 100 μl sera of patients and healthy controls serially diluted (1:250-1:2000) in PBS complemented with BSA.
(6) Remove unbound antibodies by washing 3× with 240 μl PBS/0.2% Tween 20.
(7) Bind 100 μl diluted secondary human-IgG-specific antibody fused to horseradish peroxidase (HRP).
(8) Remove unbound antibody by washing 3× with 240 μl PBS/0.2% Tween 20.
(9) Add 100 μl ortho-phenylene diamine (1 mg/ml) in PBS and stop the enzymatic reaction by adding 50 μl 4 molar sulphuric acid.
(10) Measure absorption at 490 nm.

The results are displayed in FIG. 2.

It is obvious from these data that an analysis of the binding of serum antibodies to native MOG alone allows no meaningful diagnosis whatsoever.

The antibodies from the serum of I. M. show only little binding to MOG. This would suggest that I. M. is healthy. However, this diagnosis would be incorrect, since I. M. suffers from MS.

The antibodies from the serum of N.K. show a similar behaviour as the antibodies from I.M. Note, that they were used in a concentration that was twice as high as the antibodies from I.M. This again would wrongly suggest that N.K. is healthy.

The antibodies from the serum of T. K. show a mediocre binding to MOG, about 50% more binding than the serum of I. M. Knowing that I. M. suffers from MS, one would assume that T. K. suffers from MS, too. Again, this diagnosis would be wrong because T. K. is healthy.

In contrast, however, if one considers the ratio of anti-MOG-antibodies bound to native MOG compared to anti-MOG-antibodies bound to mutant MOG, one can clearly see, that this ratio is >1 for both patients suffering from MS and <1 for the healthy control. This result is obtained independently from the type of mutant MOG used and also independently from the life circumstances of the tested individuals. Consequently, contrary to the methods of the prior art, the method of the present invention allows a safe and precise diagnosis.

Moreover, the ratio of anti-MOG-antibodies bound to native MOG compared to anti-MOG-antibodies bound to double mutant 1 is for both MS patients about 1.2, independently from the individual influences on the immune system of both patients. Similarly, the ratio of anti-MOG-antibodies bound to native MOG compared to anti-MOG-antibodies bound to double mutant 1 is for both MS patients about 1.4, despite the very different absolute amount of binding to MOG.

Consequently, for double mutant 1 a ratio of anti-MOG-antibodies bound to native MOG compared to anti-MOG-antibodies bound to double mutant 1 of about 1.2 allows to diagnose MS.

Similarly, for double mutant 2 a ratio of anti-MOG-antibodies bound to native MOG compared to anti-MOG-antibodies bound to double mutant 2 of about 1.4 allows to diagnose MS.

It is important to notice that these figures only depend on the specific mutant antibody used and that they are independent from the particular life circumstances of the tested individuals.

These examples demonstrate that the present inventors were able to provide a fast, simple' and easy-to-use method for a quantitative in vitro analysis to diagnose, to categorise, to predict and/or to monitor the progression of a condition based on antibody-antigen interactions, that overcomes or at least reduces the problems associated with the methods of the prior art, in particular that overcomes or at least reduces the problems associated with the individual properties of each subject to be analysed, in particular the amounts and kinds of antibodies present in its immune system and its incomparability with other subjects and have, hence solved the object of the present invention.

1. A method for a quantitative in vitro analysis to diagnose, to categorise, to predict and/or to monitor the progression of a condition comprising the following steps:
a) Obtaining a sample suspected of containing anti-A-antibodies from a subject to be analysed,
b) Providing native and mutant antigen A,
c) Contacting the sample suspected of containing anti-A-antibodies with mutant antigen A and with native antigen A,
d) Detecting the amount of anti-A-antibodies bound to native antigen A after step c)
wherein the presence of anti-A-antibodies bound to native antigen A allows the diagnosis, the categorisation, the prediction and/or the monitoring of the progression of a condition.

2. A method in accordance with claim 1 comprising the following steps:
a) Obtaining a first sample suspected of containing anti-A-antibodies from a subject to be analysed,
b) Providing the native antigen A,
c) Contacting the first sample suspected of containing anti-A-antibodies with the native antigen A,
d) Detecting the amount of bound anti-A-antibodies after step c),
e) Providing mutant antigen A,
f) Obtaining a second sample suspected of containing anti-A-antibodies from the same subject to be analysed as in step a),
g) Contacting the second sample suspected of containing a anti-A-antibodies from the same subject as in step a) with mutant antigen A,
h) Detecting the amount of bound anti-A-antibodies after step g),
i) Determining the ratio and/or the difference of anti-A-antibodies bound to antigen A of step d) compared to anti-A-antibodies bound to mutant antigen A of step h),
wherein the ratio and/or the difference of anti-A-antibodies bound to antigen A compared to anti-A-antibodies bound to mutant antigen A allows the diagnosis, the categorisation, the prediction and/or the monitoring of the progression of a condition.

3. Method in accordance with claim 1, further comprising the step of providing the antigen A and/or the mutant antigen A with at least one detectable moiety.

4. Method in accordance with claim 3, wherein the detectable moiety is selected from the group consisting of radioactive markers, enzymes, such as alkaline phosphatase or horseradish peroxidase, colloidal gold, urease, fluorescein, rhodamine and biotin-streptavidin.

5. Method in accordance with claim 1, wherein the individual steps are carried out in an immuno-absorbance essay, in particular in an ELISA, RIA, BIACORE or an EIA assay, preferably in an automated form.

6. Method in accordance with claim 1, wherein the condition to be diagnosed, categorised and/or its progression to be predicted and/or monitored is a physiological or a clinical condition, in particular cancer, an infectious disease, the status of a vaccination or an auto immune disorder.

7. Method in accordance with claim 1, wherein the condition to be diagnosed, categorised and/or its progression to be predicted and/or monitored is EAE and/or MS.

8. Method in accordance with claim 1, wherein a ratio of anti-A-antibodies bound to antigen A to anti-A-antibodies bound to mutant antigen A of >1, allows the diagnosis of the condition.

9. Method in accordance with claim 1, wherein the sample suspected of containing anti-A-antibodies from a subject is immobilised on a matrix prior to the contact with the antigen A and/or mutant antigen A.

10. Method in accordance with claim 1, wherein the native antigen A and/or the mutant antigen A are immobilised on a matrix prior to the contact with the sample suspected of containing anti-A-antibodies from a subject.

11. Method in accordance with claim 1, further comprising the step of contacting the anti-A-antibody-antigen A complexes after step c) and/or the step of contacting the anti-A-antibody-mutant antigen A complexes after step g) with a secondary antibody binding antibody.

12. Method in accordance with claim 1, further comprising the step of contacting the anti-A-antibody-antigen A complexes after step c) and/or the step of contacting the anti-A-antibody-mutant antigen A complexes after step g) with a secondary antigen A-binding antibody.

13. Method in accordance with claim 11, wherein the secondary antibody binding antibody and/or the secondary antigen A-binding antibody contains a detectable moiety.

14. Method in accordance with claim 8, wherein the matrix is a membrane, a cell membrane, a chip, a dish, an ELISA well, a tube, in particular a plastic or a glass tube, a cuvette, a polymer particle, a bead, a pellet or a resin for a chromatographic column.

15. Method in accordance with claim 1, wherein the sample is a blood sample, a cerebrospinal fluid sample, a serum sample or a CNS sample of a patient.

16. Method in accordance with claim 1, wherein the amount of bound antibodies is detected by visual or automated detection by spectrometry.

17. Method in accordance with claim 1, wherein the antigen A and/or the mutant antigen A is provided from a recombinant expression system.

18. Method in accordance with claim 1, wherein the native antigen A and/or the mutant antigen A is used in a refolded form.

19. Method in accordance with claim 1, wherein the native antigen A is Ro, La, Jo-1, SM, Scl70, SS-A, SS-B, Pr3, MPO, thyroglobulin, TPO, thyrotropin receptor, insulin, insulin receptor, GAD, DNA topoisomerase II, IA-2, IA-2beta, TSH receptor, PM/Scl100, acetyl choline receptor, BP180, NC1, Histone, U1 RNP, tissue transglutaminase, type IV collagen or comprises antigenic domains of these antigens or comprises antigenic parts of these antigens that share an amino acid sequence homology with the complete native antigen sequence of at least 10% identical amino acids.

20. Method in accordance with claim 1, wherein the mutant antigen A comprises at least one altered amino acid with respect to the native antigen A sequence that is located within an epitope of the native antigen A.

21. Method in accordance with claim 1, wherein the native antigen A is Myelin Oligodendrocyte Glycoprotein (MOG) or comprises antigenic parts of MOG that share an amino acid sequence homology with the native MOG sequence of at least 10 identical amino acids.

22. Method in accordance with claim 1, wherein the mutant antigen A is MOG or an antigenic part of MOG that shares an amino acid sequence homology with the native MOG sequence of at least 10 identical amino acids where at least one amino acid is altered with respect to the native MOG sequence.

23. Method in accordance with claim 1, wherein the subject to be analysed is a mammal.

24. Method in accordance with claim 2 further comprising the step of adding mutant antigen A to the sample that is brought into contact with native antigen A.

25. Method in accordance with claim 24, wherein the mutant antigen A that is added to the sample is bound on a matrix.

26. Kit for a carrying out the method of claim 1 to diagnose, to categorise, to predict and/or to monitor the progression of EAE and/or MS comprising
a) native MOG or antigenic parts of MOG that share an amino acid sequence homology with the native MOG sequence of at least 10 identical amino acids;
b) mutant MOG or an antigenic part of MOG that shares an amino acid sequence homology with the native MOG sequence of at least 10 identical amino acids;
where at least one amino acid is altered with respect to the native MOG sequence.

27. Kit in accordance with claim 26, further comprising a secondary antibody-binding antibody.

28. Kit in accordance with claim 26, further comprising a secondary MOG binding antibody.

29. Kit in accordance with claim 26, further comprising a detectable unit linked or to be linked to the native MOG and/or mutant MOG and/or secondary antibody-binding antibody and/or secondary MOG binding antibody.

30. Kit according to claim 26 further comprising a matrix to immobilize either the antigens or the antibodies.

31. Kit in accordance with claim 26, wherein at least one of the antigens or antibodies is provided in a lyophilised or denatured form.

32. Kit in accordance with claim 31, further comprising a refolding solution to refold the at least one denatured antibody or antigen.

33. Kit in accordance with claim 26, further comprising a washing solution.
